(19) United States
(12) Patent Application Publication
(10) Pub. No.: US 2011/0305728 A1
(43) Pub. Date: Dec. 15, 2011

(54) DRUG FOR TREATING INFECTIONS

(76) Inventor: Marie-Christine Etienne, Geneva (CH)

(21) Appl. No.: 12/998,743

(22) PCT Filed: Oct. 21, 2009
(86) PCT No.: PCT/IB2009/007201

§ 371 Date: Aug. 26, 2011

(30) Foreign Application Priority Data
Oct. 31, 2008 (CH) 170708

(51) Int. Cl. A61K 39/02; A61P 37/04; A61P 31/04; A61P 31/12; A61K 38/16; A61P 31/00

(52) U.S. Cl. 424/234.1; 424/282.1

(57) ABSTRACT

The invention relates to the use of glycoproteins extracted from the Klebsiella Pneumoniae bacterium for the production of a drug for the remedial treatment of acute or chronic infections. Said glycoproteins work by causing a quick lysis of all types of pathogens by means of very intense stimulation of all of the short-term, maximum 12 to 24 hour, anti-infective defense systems of the body. Said glycoproteins have proven the effectiveness thereof in 90% of common acute and chronic ENT infections, particularly to in chronic otitis which, in the prior art, often progressed inevitably towards hearing loss. Said glycoproteins have proven the effectiveness thereof in many other infections in which the bacteria or viruses do not have a high pathogenicity.

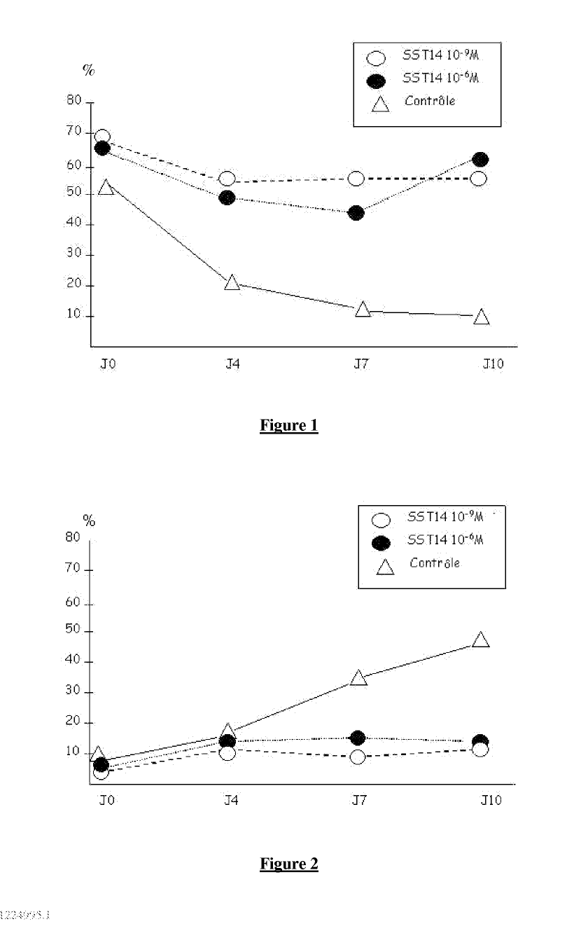

The present invention relates to the use of glycoproteins extracted from the Klebsiella Pneumoniae bacterium for the production of a drug for the remedial treatment of acute or chronic infections.

These are glycoproteins extracted from Klebsiella Pneumoniae, characterized in that they are retained on average porosity membrane 0.011 micrometer, at a dose of 1 mg expressed in anhydrous product, per tablet, pouch, capsule or any other therapeutic vector.

These are membrane fractions of Klebsiella Pneumoniae characterized in that they are to proteoglycans of Klebsiella Pneumoniae at a dose of 1.125 mg per tablet, pouch, capsule or any other therapeutic vector.

These membranous proteoglycans can be used alone or associated with bacterial ribosomes titrated at 70% of RNA characterized in that for 1.125 mg of membranous proteoglycans, there are 0.750 mg of bacterial ribosomes, broken down as follows: 0.2625 mg of ribosomes from Klebsiella Pneumoniae, 0.225 mg of ribosomes from Streptococcus Pneumoniae, 0.225 mg of ribosomes from group A pyogenic Streptococcus, 0.0375 mg of ribosomes from Haemophilus Influenzae.

In view of the low content in active ingredients, these glycoproteins are accompanied, within the drug, by excipients to enhance their distribution inside the organism:

In order to produce a tablet, an excipient such as defined hereafter will preferably be used, including: lactose, cornstarch, magnesium stearate, cellulose acetyl phthalate, dibutylphthalate, saccharose, sodium carmellose, colloidal silica, talcum, polyoxyethylene (20) sorbitan mono-oleate (polysorbate 80), titanium dioxide, white wax.

In order to produce a tablet, another type of excipient will preferably be used, including: aerosil, magnesium stearate and sorbitol.

Any other type of excipient enhancing the diffusion of the glycoproteins can be used.

The invention is characterized in that these glycoproteins as defined above will be used at the dose of two tablets per day, at a dosage of one tablet mornings and evenings for a person weighing up to 50 kg; above that weight, it is necessary to slightly increase the doses to prevent the effect from diminishing. It is possible to reduce these doses or to increase them in order to adapt the treatment to the pathological condition.

In the prior art, these glycoproteins extracted from the Klebsiella Pneumoniae bacterium as described above have been marketed under the name Biostim and Ribomunyl in France. In the prior art, they were used in the preventive treatment of recurring infections of the ENT organs and of the bronchi, which is the indication for which these products were granted a marketing authorization.

It was known in the prior art that these weak doses of glycoproteins were capable of reinforcing the immune defenses, especially if these were deemed deficient, and of increasing the resistance of the organism to infections occurring subsequently to their intake. They were taken over several weeks interspaced with treatment time windows.

The invention relates to a surprising and unexpected property of these glycoproteins, namely that if they are taken at the beginning of an infection, these glycoproteins cause a quick lysis of all types of pathogens by means of very intense stimulation of all of the short-term, maximum 12 to 24 hour, anti-infective defense systems of the body and, accordingly, a cure of the infection (which requires the intake to be repeated every 12 hours).

These glycoproteins cause, in these conditions, an intense stimulation of the entire immune system, with a sharp increase of all immuno-competent cells, a sharp increase of the cells destined to destroy pathogens, a sharp increase of all the antibodies including the IgM, IgG and IgA that are to destroy the pathogens. They also increase all anti-infective mediators.

All pathogens, whatever their nature, can thus be destroyed by the action of these glycoproteins: bacteria, viruses, mycoses and others such as bacilli, prions, etc.

In the prior art, it is necessary in order to treat infections to use products adapted to the pathogen and to the site of the infection: antibiotic for bacteria, antiviral for viruses, antifungal for mycoses. Used at doses 100 or 1,000 times greater than those of glycoproteins, i.e. 2 mg per day, these preparations cause tiredness and reactions of intolerance, which is not the case with these glycoproteins that do not give the impression of having been taken as a drug and thus enjoy an exceptional tolerance.

In a surprising and unexpected manner, these glycoproteins have been shown to cure a great number of very common acute infections that are not of immediately serious nature, in particular ENT infections and chronic infections, in particular chronic infections that persist after antibiotic treatment.

Among the acute infections that are particularly concerned by the invention one can mention preferably:
ENT infections: rhinitis, rhinopharyngitis, amygdalitis, otitis , sinusitis, etc.
Digestive infections: gastro-enteritis, gastric infections, intestinal infections, etc.
Respiratory infections: laryngitis, tracheitis, bronchitis, etc.
Genito-urinary infections: cystitis, vaginitis, pelvic infections, etc.

The invention relates to all chronic infections without limitation and in particular to to chronic infections that persist after antibiotic treatment, in particular chronic otitis.

If most of the time, these glycoproteins used alone have proven their effectiveness in this type of acute or chronic infections, their association with other bacterial extracts can increase their effectiveness. These extracts can be bacterial ribosomes or antigenic bacterial lysates belonging to various preparations such as: IRS19, Rhinopten, Imudon, Lantigen B, intestinal or urinary ampho-vaccine . . . .

The invention relates in particular to contagious respiratory diseases that occur most frequently in winter, which are due in part to viruses and in part to bacteria and which start with a febrile rhinopharyngitis and evolve into bronchitis over 10 days, causing a temperature rise over the entire progression.

This treatment using glycoproteins extracted from Klebsiella Pneumoniae was effective to cure in 2 to 3 days a considerable number of these respiratory affections at the rhinopharyngitis stage, thus preventing the evolution into bronchitis that regularly occurred before. This glycoprotein treatment according to the invention was effective to cure a great number of cases of catarrh of the Eustachian tube as well as a great number of cases of congestive otitis.

This glycoprotein treatment according to the invention has cured a chronic otitis that persisted after 3 weeks of antibiotics with remanent temperature, dull earache and recent apparition of tinnitus, bearing witness to the infection's progression to the auditory organ that is located in the mastoid which was painful. One month of treatment with glycoproteins was necessary to clear all these symptoms. The tinnitus disappeared within the first days of this treatment. The dosage regimen was, as always, one tablet mornings and evenings. There were treatment time windows once per week.

The treatment with glycoproteins extracted from Klebsiella Pneumoniae was effective to cure conjunctivitis attacks with considerable conjunctival suppuration, which resisted to antibiotic and antiseptic collyria and was feared to progress to a serious infectious keratitis or deep eye infection with risk of eyesight loss. From the first intake of glycoprotein, the quantity of conjunctival pus diminished and so did the associated low-grade fever with associated rhinitis. Eight days of treatment with glycoproteins were necessary to completely cure these conjunctivitis symptoms. The clinical form evoked a to viral origin of this conjunctivitis: adenovirus, RSV.

Several attacks of stye were cured in 3 to 5 days with glycoprotein treatment.

Several attacks of mouth aphtha reacted to this glycoprotein treatment according to the invention, including a spectacular diminution of the aphtha in question within hours of the first intake of glycoprotein. The treatment lasted 3 to 4 days.

Several infectious attacks of the outer hearing canal have successfully been treated with the glycoproteins according to the invention, with a clear diminution of earache, of pruritus, of associated signs such as slight temperature or light hearing impairment, from the first intake of said glycoproteins. A treatment over 5 to 6 days was required.

These glycoproteins extracted from Klebsiella Pneumoniae proved effective to cure benign gastroenteritis including possibly infections with Helico Bacter Pylori. From the first intake of these glycoproteins, the nausea and vomiting, if any, clearly disappeared and there was an improvement of the overall condition: lessening of tiredness and of low-grade temperature, if there was any, and alleviating of associated symptoms if there were any: rhinitis, headaches, diarrhea. This treatment lasted 2 days on average.

This glycoprotein treatment according to the invention was effective to cure abdominal pain persisting after various infections: digestive or gynecological after antibiotic treatment such as sigmoiditis or pelvic chlamydiosis. One or two weeks of glycoprotein treatment were necessary.

The treatment using glycoproteins extracted from Klebsiella Pneumoniae proved effective to overcome persisting signs of cystitis after antibiotic treatment, such as pollakiuria, burning sensation when urinating, and to curb new relapses of such cystitis with the same symptoms.

These glycoproteins have proven their effectiveness in viral infections such as herpes zoster (shingles) where the eruption disappeared within 3 days with a treatment using these glycoproteins with, from the first intake of these glycoproteins, a lessening of the low-grade fever condition and improvement of the general condition.

A treatment using the glycoproteins according to the invention cured an attack of persisting hacking cough that resisted to antibiotic treatment and which had occurred after contact with a child affected with whooping cough.

An 8-day treatment with these glycoproteins coincided with the disappearing of a carcinogen papillomavirus observed on a swab test and which was no longer present on to the next swab test. Although no conclusion can be drawn regarding a possible ailment, it can however be considered that these glycoproteins are capable of having the organism eliminate carcinogen viruses and other microorganisms responsible for cancer such as Helico Bacter Pylori.

These glycoproteins extracted from Klebsiella Pneumoniae have proven effective in case of contact with a contagious patient to prevent an affliction from occurring: one or two days of treatment were necessary.

Several cough attacks with expectorations persisting for a certain time after antibiotic treatment have been cured by taking these glycoproteins during 8 days on average. One of these attacks was strongly reminiscent of an infection by the Respiratory Syncitial Virus (RSV).

The failure rate of the treatment using these glycoproteins was 8%. These were cases of rhinopharyngitis that progressed to bronchitis despite this glycoprotein treatment. There was then an increased inflammatory reaction of the bronchi with a small increase of sputum volume. It is probable that the great virulence of the infectious agent thwarted the organism's ability to resist and to mobilize all its antibodies. It is also possible that the reason for the failure is to be found in the absence of prior contact with the infectious agent so that no memory cells could be mobilized. In the failure cases, the antibiotic treatment was much shortened as compared with what it would have been without prior treatment with these glycoproteins and lasted only two days on average.

It is thus possible to conclude that glycoproteins extracted from the Klebsiella Pneumoniae bacterium are very effective for treating infections that are not of immediately serious nature, i.e. in the absence of high pathogenicity. It is possible to conclude that these glycoproteins are effective for treating infections in their early stages, very frequent in the winter, as well as for treating chronic infections persisting after antibiotic treatment, notably for treating chronic otitis which, in the prior art, often progressed inevitably towards serious hearing loss.

It can thus be concluded that this new use of glycoproteins extracted from Klebsiella Pneumoniae is very promising as regards industrial application and commercialization.

They are in fact the best-tolerated products due to their low active substance contents. They are liable to suit 96% of the population for a frequent use. By having the pathogens destroyed, their first effect is to bring fever down. In the cases of failure when bronchitis developed, they always prevented otitis from occurring.

The 4% of the population that will not benefit from this new use of these glycoproteins to are those who have counter-indications: by increasing the proportion of all antibodies, these glycoproteins also increase that of those antibodies that are responsible for allergy and auto-immunity (IgE for allergies). Benign allergies such as allergic rhinitis or limited eczemas are not counter-indications: their worsening is reversible in that the initial state is reverted to within 24 hours after stopping use of these glycoproteins. Serious allergies such as asthma are counter-indications to use of these glycoproteins. It is necessary to be cautious for patients that have pre-existing allergic conditions and bear various prosthetics or intraocular prosthetic lenses. Retrovirus viral infections must prompt caution because retrovirus infect immuno-competent cells and by having this type of cell multiply, there is a risk of having infected cells and thus the viruses multiply. This hypothesis has however not been studied during assays. These retrovirus infections are AIDS, lymphosarcoma, lymphoma. All types of leukemia and leucosis are counter-indications from this perspective.

The pharmaceutical industry has abandoned use of these glycoproteins extracted from Klebsiella Pneumoniae, withdrawing all of them from the market in 2006, totally unaware of the surprising and unexpected properties of these glycoproteins that are the object of the present invention. This industry preferred refocusing and holding on to products designed to bring down fever such as aspirin and paracetamol as well as products designed to destroy pathogens such as antibiotics and antivirals, without suspecting for one moment that these glycoproteins could have these properties.

1. A method of treating an acute or chronic infection in a subject, the method comprising administering to the subject glycoproteins extracted from Klebsiella Pneumoniae bacterium, wherein the glycoproteins can be retained on average porosity membrane 0.011 micrometer and are administered at a dose of 1 mg per tablet or pouch; or administering membranous proteoglycans extracted from Klebsiella Pneumoniae bacterium at a dose of 1.125 mg per tablet or capsule, for an average posology of 2 per day.

2. A method of treating an infection of the whole organism in a subject, the method comprising administering glycoproteins extracted from Klebsiella Pneumoniae bacterium to the subject.

3. The method according to claim 1, wherein the acute or chronic infection is caused by a pathogen selected from the group consisting of bacteria, viruses, bacilli, prions and carcinogens.

4. The method according to claim 1 wherein the glycoproteins are administered with either antigenic lysates from bacteria or with ribosomes from bacteria titrated at 70% of RNA.

5. The method according to claim 1 wherein the glycoproteins all have the same chemical formula or have different chemical formula.

6. The method according to claim 1 wherein the glycoproteins cause a short stimulation of the subject's immune system, wherein the immune system stimulation causes an increase in immuno-competent cells, an increase in cells destined to destroy pathogens, and an increase in antibodies and anti-infective mediators,

7. The method according to claim 1, wherein the infection is an acute infection selected from the group consisting of an ENT infection, a pulmonary infection, an ophthalmological infection, an intestinal infection and a genito-urinary infection.

8. The method according to claim 1, wherein the infection is a chronic infection selected from the group consisting of an ENT infection, a pulmonary infection, an ophthalmological infection, an intestinal infection and a genito-urinary infection.

9. The method according to claim 1, wherein the acute or chronic infection persists after antibiotic treatment in the subject.

10. The method according to claim 2, wherein the glycoproteins are administered with either antigenic lysates from bacteria or with ribosomes from bacteria titrated at 70% of RNA.

11. The method according to claim 1, wherein the glycoproteins are administered to the subject with a further active agent.

12. The method according to claim 11, wherein the glycoproteins and further active agent are within the same pharmaceutical dosage form.

13. The method according to claim 2, wherein the glycoproteins all have the same chemical formula or have different chemical formula.

14. The method according to claim 2, wherein the glycoproteins are administered to the subject with a further active agent.

15. The method according to claim 14, wherein the glycoproteins and further active agent are within the same pharmaceutical dosage form.

16. The method according to claim 6, wherein the short stimulation of the subject's immune system does not exceed 12 to 24 hours, and wherein the glycoproteins are administered every 12 hours.

17. The method according to claim 7, wherein the acute infection is in the initial phase.

18. The method according to claim 17, wherein the acute infection is otitis or a respiratory infection starting with rhinopharyngitis.

19. The method according to claim 8, wherein the chronic infection is otitis.
